(19) United States
(12) Patent Application Publication
(10) Pub. No.: US 2024/0376434 A1
(43) Pub. Date: Nov. 14, 2024

(54) CELL EXPANSION METHODS AND COMPOSITIONS FOR USE THEREIN

(71) Applicants: LIFE TECHNOLOGIES CORPORATION, Carlsbad, CA (US); NUNC A/S, Roskilde (DK)

(72) Inventors: Debra JOHNSON, San Marcos, CA (US); Sune SPORRING, Naestved (DK)

(21) Appl. No.: 18/690,262

(22) PCT Filed: Sep. 16, 2022
(86) PCT No.: PCT/US2022/076593

§ 371 Date: Mar. 7, 2024

Related U.S. Application Data
(60) Provisional application No. 63/245,153, filed on Sep. 16, 2021.

(51) Int. Cl. C12N 5/0775; A61K 35/28; C12N 9/22

(52) U.S. Cl. CPC C12N 5/0663; A61K 35/28; C12N 9/22; C12N 2310/20; C12N 2500/98; C12N 2500/99; C12N 2510/00

(57) ABSTRACT

The instant technology generally relates to methods and compositions for expansion of mesenchymal stem cells in culture on a modified surface in the absence of a cell feeder layer and the absence of a cell adhesive coating on the surface. In some instances, the mesenchymal stem cells are expanded on the modified surface in a serum-free culture medium.

### CROSS-REFERENCE TO RELATED APPLICATIONS

This application claims the benefit of priority under 35 U.S.C. § 119(e) to U.S. Provisional Application No. 63/245,153, filed Sep. 16, 2021, the entire contents thereof is hereby expressly incorporated by reference as though fully set forth herein.

### BACKGROUND

Mesenchymal stem cells (MSCs) are cells with the ability to differentiate into different types of various cells, such as osteocytes, chondrocytes, fibroblasts, myocytes, epithelial cells, hepatocytes, or neurons. These multipotent cells are being clinically explored as a therapeutic for treating a variety of human diseases. Since MSCs permit autologous transplantation, they are a type of cell well-suited for cell and/or gene therapies and for applications in regenerative medicine. Immunological properties of MSCs, such as anti-inflammatory, immunoregulatory and immunosuppressive capacities, contribute to their potential role as immune tolerant agents.

MSCs exist in, and are easily extracted from, almost all tissues, including bone marrow, adipose, synovium, and umbilical cord blood. Methods for extraction, isolation, and identification of MSCs from a variety of human and animal tissues and organs are well documented in the literature. Culture media and conditions have also been described for the growth and maintenance of pluripotent MSCs as well as for multilineage differentiation of MSCs in culture.

Current methods for growing MSCs in culture require the presence of serum in culture media and/or an extra-cellular matrix protein(s) coating the cell culture surface. Some methods also call for culturing MSCs on a feeder cell layer covering the culture growth surface. Feeder cells, serum and extra cellular matrix proteins are usually undefined cell culture reagents and as such cannot be used in the production of cell therapy products or medicaments. The use of serum and/or extra cellular matrix coatings also adds to cost and introduces variability into the cell culture and production workflow. There remains a need for methods and reagents for expanding MSCs in culture, in particular to obtain MSC populations suitable for a variety of therapeutic applications.

### SUMMARY OF THE INVENTION

The instant technology generally relates to methods and compositions for expansion of mesenchymal stem cells. In aspects, the technology relates to methods and compositions for expanding mesenchymal stem cells in culture media and on a modified culture surface without a feeder cell layer or a cell adhesive coating on the surface. In embodiments, the methods comprise expanding a mesenchymal stem cell population in culture in serum-free media and on a modified culture surface without a feeder cell layer or cell adhesive coating on the surface.

In an aspect, herein is provided a method of expanding a mesenchymal stem cell population, including: (i) obtaining a plurality of mesenchymal stem cells (MSCs); and (ii) culturing the MSCs in growth medium on a surface, wherein the surface lacks a cell adhesive coating and lacks a feeder cell layer, thereby generating an expanded MSC population. In embodiments, the growth medium is serum-free. In embodiments, the growth medium is animal origin-free medium and/or xeno-free medium.

In embodiments, the surface contains at least about 0.9% N, has a sum of O and N of greater than or equal to 19.0% and has a static sessile contact angle of at least about 14 degrees. In embodiments, the surface contains about 0.9% to about 3.2% N, has a sum of O and N of about 19% to about 35% and has a sessile contact angle of about 14 to about 65 degrees. In embodiments, the surface contains at least about 0.9% N, has a sum of O and N of greater than or equal to 19.0% and has a static sessile contact angle of at least about 14 degrees. In embodiments, the surface contains about 0.9% to about 3.2% N, has a sum of O and N of about 19% to about 35% and has a sessile contact angle of about 14 to about 60 degrees. In embodiments, the surface contains about 1.3% to about 2.8% N, has a sum of O and N of about 22% to about 29% and has a sessile contact angle of about 17 to about 55 degrees.

In embodiments, the surface is modified using a plasma treatment. In embodiments, the surface comprises polystyrene, cyclic olefin polymer, cyclic olefin copolymer, polyolefin, polycarbonate, polymethyl methacrylate, styrene acrylonitrile copolymer, and mixtures and copolymers therefrom. In embodiments, the surface is part of a vessel or matrix.

In embodiments, the MSCs are isolated from bone marrow, adipose tissue, dermis, placenta, umbilical cord, amniotic fluid, synovial fluid, synovial membrane, deciduous teeth, or skeletal muscle. In embodiments, the MSCs are human MSCs, non-human primate MSCs, murine MSCs, rat MSCs, pig MSCs, goat MSC, or sheep MSCs.

In embodiments, culturing the MSCs includes growing the MSCs in growth medium on the surface through at least 3 passages. In embodiments, culturing the MSCs includes growing the MSCs in growth medium on the surface through at least 6 passages.

In embodiments, the expanded MSC population is incubated under conditions allowing the differentiation of the MSCs. In embodiments, the expanded MSC population is incubated under conditions allowing the differentiation of the MSCs into cells selected from the group consisting of osteoblasts, adipocytes, and chondrocytes.

In an aspect, herein is provided a method for editing a genome in an MSC, including: (i) obtaining an MSC that was expanded using an expansion method provided herein; and (ii) editing the genome of the MSC. In embodiments, the genome is edited using one of more genome editing reagents selected from a zinc-finger nuclease (ZFN), transcription activator-like effector nuclease (TALEN), meganuclease, and a clustered regularly interspaced short palindromic repeat (CRISPR) associated protein.

In an aspect, herein is provided a method of treating a subject in need of a therapy, including: (i) obtaining a plurality of mesenchymal stem cells (MSCs); (ii) expanding the MSCs in serum-free growth medium on a surface, wherein the surface lacks a cell adhesive coating and lacks a feeder cell layer; and (iii) transferring the expanded MSCs to the subject, thereby treating the subject. In embodiments, the surface contains at least about 0.9% N, has a sum of O and N of greater than or equal to 19% and has a static sessile contact angle of at least about 14 degrees. In embodiments, the plurality of MSCs are derived from the subject. In embodiments, the method further includes genetically modifying the expanded MSCs prior to transferring the MSCs to the subject.

In an aspect, herein is provided a method for improving engraftment potential of a population of mesenchymal stem cells (MSCs), including: (i) obtaining a plurality of mesenchymal stem cells (MSCs); and (ii) expanding the MSCs in serum-free growth medium on a surface containing: at least about 0.9% N, a sum of O and N of greater than or equal to 19% and having a static sessile contact angle of at least about 14 degrees, wherein the surface lacks a cell adhesive coating and lacks a feeder cell layer; thereby improving engraftment potential of the population of MSCs.

In an aspect, herein is provided use of an expanded population of MSCs generated according to an MSC expansion method provided herein for the preparation of a medicament.

In an aspect, herein is provided a population of mesenchymal stem cells (MSCs), expanded using an MSC expansion method provided herein for use in a medicament to treat a subject. In embodiments, the MSCs are derived from the subject to be treated prior to expansion. In embodiments, the MSCs of the expanded population are genetically modified.

### BRIEF DESCRIPTION OF THE DRAWINGS

FIGS. 1A-F are graphs depicting cell growth (as measured by population doublings) over time and cell viability of MSCs growing in serum-free medium on culture surfaces with or without coating. FIGS. 1A and 1B show growth and viability of adipose-derived MSCs (ADSC). FIGS. 1C and 1D show growth and viability of bone marrow-derived MSCs (BMMSC). FIGS. 1E and 1F show growth and viability of Wharton Jelly-derived MSCs (WJMSC).

FIG. 2 shows micrographs of adipose-derived MSCs cultured in serum-free medium on different surfaces: Delta surface without coating (first column), Delta surface with coating (second column), Surface A without coating (third column), Surface A with coating (fourth column). Representative micrographs of the cell cultures at passages 2-4 are shown (scale bar=275 μm). The cells did not survive past passage 2 when grown on the uncoated Delta surface in serum-free conditions.

FIG. 3 shows micrographs of bone marrow-derived MSCs cultured in serum-free medium on different surfaces: Delta surface without coating (first column), Delta surface with coating (second column), Surface A without coating (third column), Surface A with coating (fourth column). Representative micrographs of the cell cultures at passages 4-6 are shown. The cells did not attach well for growth when grown on the uncoated Delta surface in serum-free conditions.

FIG. 4 shows micrographs of Wharton's Jelly-derived MSCs cultured in serum-free medium on different surfaces: Delta surface without coating (first column), Delta surface with coating (second column), Surface A without coating (third column), Surface A with coating (fourth column). Representative micrographs of the cell cultures at passages 4-6 are shown. The cells did not survive past passage 3 when grown on the uncoated Delta surface in serum-free conditions.

FIGS. 5A-5X show graphs depicting cell surface marker expression as determined by flow cytometry analysis of adipose-derived human MSCs cultured in serum-free medium on different surfaces: Delta surface with coating (FIGS. 5A-5H), Surface A without coating (FIGS. 5I-5P), Surface A with coating (FIGS. 5Q-5X). Cell surface marker expression shown: CD90 (FIGS. 5A, 5I, 5Q), CD73 (FIG. 5B, 5J, 5R), CD105 (FIGS. 5C, 5K, 5S), CD44 (FIGS. 5D, 5L, 5T), CD34 (FIGS. 5E, 5M, 5U), CD45 (FIGS. 5F, 5N, 5V), CD14 (FIGS. 5G, 50, 5W), CD79a (FIGS. 5H, 5P, 5X). The x-axis is a log scale and the numbers are the exponent of 10.

FIGS. 6A-6O show graphs depicting cell surface marker expression as determined by flow cytometry analysis of bone marrow-derived human MSCs cultured in serum-free medium on different surfaces: Delta surface with coating (FIGS. 6A-6E), Surface A without coating (FIGS. 6F-6J), Surface A with coating (FIGS. 6K-6O). Cell surface marker expression shown: CD90 (FIGS. 6A, 6F, 6K), CD73 (FIG. 6B, 6G, 6L), CD105 (FIGS. 6C, 6H, 6M), CD44 (FIGS. 6D, 6I, 6N). The graphs of markers CD34, CD45, CD14 and CD79 were combined (FIGS. 6E, 6J, 6O). The x-axis is on log scale and the numbers are the exponent of 10.

FIGS. 7A-7X show graphs depicting cell surface marker expression as determined by flow cytometry analysis of Wharton's Jelly-derived human MSCs cultured in serum-free medium on different surfaces: Delta surface with coating (FIGS. 7A-7H), Surface A without coating (FIGS. 7I-7P), Surface A with coating (FIGS. 7Q-7X). Cell surface marker expression shown: CD90 (FIGS. 7A, 7I, 7Q), CD73 (FIG. 7B, 7J, 7R), CD105 (FIGS. 7C, 7K, 7S), CD44 (FIGS. 7D, 7L, 7T), CD34 (FIGS. 7E, 7M, 7U), CD45 (FIGS. 7F, 7N, 7V), CD14 (FIGS. 7G, 70, 7W), CD79α (FIGS. 7H, 7P, 7X). The x-axis is a log scale and the numbers are the exponent of 10.

FIG. 8 shows micrographs of bone marrow-derived MSCs differentiated to chondrocytes (stained with Alcian Blue top row), osteocytes (stained with Alizarin Red S, middle row) and adipocytes (stained with Oil Red O, bottom row). The bone marrow-derived MSCs were cultured in serum-free medium on: Delta surface with coating (left column), Surface A without coating (middle column), and Surface A with coating (right column).

FIG. 9 shows micrographs of adipose-derived MSCs differentiated to adipocytes (stained with Oil Red O, top row), osteocytes (stained with Alizarin Red S, middle row) and chondrocytes (stained with Alcian Blue, bottom row). The bone marrow-derived MSCs were cultured in serum-free medium on: Delta surface with coating (left column), Surface A without coating (middle column), and Surface A with coating (right column). Scale bar=275 μm.

FIG. 10 shows micrographs of Wharton's Jelly-derived MSCs differentiated to adipocytes (stained with Oil Red O, top row), osteocytes (stained with Alizarin Red S, middle row) and chondrocytes (stained with Alcian Blue, bottom row). The bone marrow-derived MSCs were cultured in serum-free medium on: Delta surface with coating (left column), Surface A without coating (middle column), and Surface A with coating (right column).

### DETAILED DESCRIPTION

Described herein are improved methods and compositions for expanding mesenchymal stem cells in culture without an extracellular matrix or other cell adhesion coating on culture surfaces. The technology provided herein permits the production of large numbers of viable, pluripotent MSCs using defined culture reagents and cell therapy grade materials. The cell expansion environment can also be essentially free of feeder cells since feeder cells are not required to keep the MSCs proliferating in an undifferentiated state.

In one aspect, the present disclosure provides methods of expanding a mesenchymal stem cell population by culturing the MSCs in growth medium on a modified surface lacking a coating that promotes cell adhesion and lacking a feeder cell layer. A method of expanding an MSC population includes culturing the MSCs in a serum-free growth medium on the modified surface without a coating or feeder cell layer.

Described herein are methods of expanding a mesenchymal stem cell population by culturing the cells in growth medium on a modified surface containing at least about 0.9% N, a sum of O and N of greater than or equal to 19% and a contact angle of at least about 14 degrees, and lacking a coating that promotes cell adhesion and lacking a feeder cell layer. Also described herein are methods of expanding a mesenchymal stem cell population by culturing the cells in serum-free growth medium on the modified surface without a coating or feeder cells.

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Optional” or “optionally” means that the subsequently described event or circumstance can or cannot occur, and that the description includes instances where the event or circumstance occurs and instances where it does not.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The terms “serum-free,” and “lacking serum” as used herein refer to medium which is free or substantially free of serum. “Substantially free of serum” as used herein refers to media which contains less than about 1% serum by weight, contains only trace amounts of serum, or contains undetectable amounts of serum.

The term “chemically-defined medium” as used herein refers to medium suitable for in vitro culture of cells, particularly eukaryotic cells, in which all of the chemical components and their concentrations are known.

The phrase “protein-free” culture medium refers to culture medium that contain no protein (e.g., no serum proteins such as serum albumin or attachment factors, nutritive proteins such as growth factors, or metal ion carrier proteins such as transferrin, ceruloplasmin, etc.). Preferably, if peptides are present, the peptides are smaller peptides, e.g., di- or tri-peptides. Preferably, peptides of deca-peptide length or greater are less than about 1%, more preferably less than about 0.1%, and even more preferably less than about 0.01% of the amino acids present in the protein free medium.

The term “animal derived” material as used herein refers to material that is derived in whole or in part from an animal source, including recombinant animal DNA or recombinant animal protein DNA.

The phrases “cell culture medium,” “growth medium,” “tissue culture medium,” “culture medium” (plural “media” in each case) and “medium formulation” refer to a nutritive solution for cultivating cells or tissues. These phrases can be used interchangeably.

The terms “basal medium,” “basal cell culture medium,” “basal culture medium” and the like as used herein refer to a cell culture medium that contains amino acids, vitamins, inorganic salts, and at least one carbon source. In some instances, basal media formulations must be further supplemented with a supplement, a chemically-defined medium and/or serum (or similar additive). Supplemented basal media are suitable for growth of various types of cells. As used herein, the terms “supplement” or “supplement composition” refer to a composition when added to a basal medium may be beneficial for cell maintenance, expansion, growth, and viability, or may increase cell proliferation, may maintain pluripotency, may increase passage count, may increase culture scale or the like.

By “cultivation” is meant the maintenance of cells in vitro under conditions favoring growth, expansion, and/or differentiation and/or or continued viability. “Cultivation” can be used interchangeably with “cell culture.” Cultivation is assessed by number of viable cells/mL culture medium.

By “expansion” is meant the growth of cells in culture to increase the number of the cells from an initial cell number to a larger cell number following time in culture. The cell number may include, without limitation, total cells, total viable cells, total nucleated cells, or any combination thereof.

In embodiments, cell growth or cell population expansion is measured by doubling time. “Doubling time” is the period of time required for the cell number in the culture to double. Doubling time can be determined by counting the number of cells in culture at two or more time points during culture of the cells. In embodiments, doubling time is calculated by the following formula:

$$DoublingTime = \frac{duration * log ( 2 )}{log ( FinalConcentration ) - log ( InitialConcentration )} .$$

As used herein, “maintenance” refers generally to cells placed in a growth medium under conditions that facilitate cell growth and/or division that may or may not result in a larger population of the cells.

As used herein, “passaging” or “subculturing” refers to the process of removing cells from one culture vessel and placing them in a second culture vessel under conditions that facilitate cell growth, expansion and/or division. In some embodiments, passaging can include dissociation of cell clusters or cell monolayers to obtain smaller clusters or individual cells, followed by growth of the dissociated clusters or cells in culture media. In some embodiments, all or a portion of the dissociated cell clusters or cells are placed in different culture media. In some embodiments, all or a portion of the dissociated cell clusters or cells are placed in different culture vessels.

A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore, the number of population doublings of a culture is greater than the passage number. The expansion of cells (i.e., the number of population doublings) during the period between passaging depends on many factors, including but not limited to the seeding density, substrate, medium, growth conditions, and time between passaging.

“Markers” as used herein, are nucleic acid or polypeptide molecules or other types of molecules that are differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

“Surface” as used herein refers to the outermost layers of molecules of a solid substrate vessel or matrix intended for use in cell culture or analysis. The elemental composition, the roughness, and the wettability of the surface can be analyzed by X-Ray Photoelectron Spectroscopy (XPS), Atomic Force Microscopy (AFM), and contact angle measurement, respectively.

“Coating” or “adhesive coating” as used herein refers to a layer that is formed on a surface of a solid substrate by attaching molecules to the surface whereby the coated molecules promote cell attachment to the surface. Molecules used in making a coating can be, without limitation, non-biological molecules, such as for example, polyethyleneimine, or biological molecules, such as for example, extracellular matrix proteins, peptides and basement membrane extract.

The present disclosure provides methods for cultivation of mesenchymal stem cells (MSCs) on a surface which has been modified to contain from at least about 0.9% N, a sum of O and N of greater than or equal to 19.0% and a sessile contact angle of at least about 14 degrees, the modified surface lacking an adhesive coating and a feeder cell layer. The present disclosure provides methods for attachment to and cultivation of MSCs on a surface in the absence of serum in culture media, the absence of a feeder cell layer and the absence of an adhesive coating on the surface, which surface has been modified to contain N in the range of about 0.9% to about 3.2%, a sum of N and O in the range of about 19% to about 35% and a contact angle of about 14 degrees to about 60 degrees.

The present disclosure provides a method of expanding a mesenchymal stem cell population, the method comprising obtaining a plurality of mesenchymal stem cells (MSCs) and culturing the MSCs in serum-free growth medium on a surface, wherein the surface lacks an adhesive coating, such as an extracellular matrix coating, and lacks a feeder cell layer, thereby generating an expanded MSC population.

The present disclosure provides a method of expanding a mesenchymal stem cell (MSC) population, the method comprising obtaining a plurality of MSCs and culturing the MSCs in growth medium on a surface containing at least about 0.9% N, a sum of O and N of greater than or equal to 19.0% and having a static sessile contact angle of at least about 14 degrees, wherein the surface lacks an adhesive coating and lacks a feeder cell layer, thereby generating an expanded MSC population. In an embodiment, the method comprises culturing the MSCs in serum-free growth medium on a surface containing at least about 0.9% N, a sum of O and N of greater than or equal to 19.0% and having a static sessile contact angle of at least about 14 degrees, wherein the surface lacks an adhesive coating and lacks a feeder cell layer.

In embodiments, the MSCs are expanded in the culture medium on a modified surface containing about 0.9% to about 3.2% N, about 1.1% to about 3.0% N, about 1.3% to about 2.8% N, about 1.5% to about 2.6% N, about 1.7% to about 2.4% N, about 0.9% to about 2.5% N, about 1.1% to about 2.5% N, about 1.3% to about 2.5% N, about 2.1% to about 3.2% N, about 1.9% to about 3.2% N, or about 1.7% to about 3.2% N.

In embodiments, the MSCs are expanded in the culture medium on a modified surface containing a sum of O and N of about 19% to about 35%, about 21% to about 33%, about 22% to about 31%, about 22% to about 29%, about 22% to about 27%, about 19% to about 29%, about 19% to about 27%, about 19% to about 25%, about 19% to about 23%, about 24% to about 35%, about 24% to about 33%, about 24% to about 31%, or about 24% to about 29%.

In embodiments, the MSCs are expanded in the culture medium on a modified surface having a sessile contact angle of about 14 degrees to about 65 degrees, about 14 degrees to about 60 degrees, about 17 degrees to about 55 degrees, about 20 degrees to about 50 degrees, about 30 degrees to about 50 degrees, about 38 degrees to about 55 degrees, about 17 degrees to about 45 degrees, about 30 degrees to about 55 degrees, about 30 degrees to about 65 degrees, or about 40 degrees to about 65 degrees.

Accordingly, the present disclosure provides a method of expanding a MSC population, the method comprising culturing the MSCs in growth medium on a modified surface which lacks an adhesive coating and lacks a feeder cell layer, where the surface contains about 0.9% to about 3.2% N, a sum of O and N of about 19% to about 35%, and has a sessile contact angle of about 14 to about 60 degrees. In some embodiments, the present disclosure provides a method of expanding a MSC population, the method comprising culturing the MSCs in growth medium on a surface which lacks an adhesive coating and lacks a feeder cell layer, where the surface contains about 1.3% to about 2.8% N, a sum of O and N of about 22% to about 29%, and has a sessile contact angle of about 17 to about 55 degrees.

The present disclosure provides a method of expanding a MSC population, the method comprising culturing the MSCs in a serum-free growth medium on a surface which lacks an adhesive coating and lacks a feeder cell layer, where the surface contains about 0.9% to about 3.2% N, a sum of O and N of about 19.0% to about 35%, and has a sessile contact angle of about 14 to about 60 degrees. In some embodiments, the present disclosure provides a method of expanding a MSC population, the method comprising culturing the MSCs in a serum-free growth medium on a surface which lacks an adhesive coating and lacks a feeder cell layer, where the surface contains about 1.3% to about 2.8% N, a sum of O and N of about 22% to about 29%, and has a sessile contact angle of about 17 to about 55 degrees.

In methods provided, the MSC population is expanded on the modified surface in serum-free growth medium through at least 2 passages. In other methods provided, the MSC population is expanded on the modified surface in serum-free growth medium through at least 3 passages. In some methods provided, the MSC population is expanded on the modified surface in serum-free growth medium through at least 4 passages, at least 5 passages, at least 6 passage, at least 7 passages, at least 8 passages, at least 9 passages, at least 10 passages, at least 11 passages, or at least 12 passages. In some methods provided, the MSC population is expanded for 2 to 6 passages or for 3 to 5 passages. In some methods provided, the MSC population is expanded for 5 to 10 passages or for 4 to 8 passages.

In some embodiments, the plurality of MSCs are cultivated on culture vessel surface having a coating that promotes cell adhesion for a period of time prior to the culturing on the modified surfaced without a coating. In some of the methods provided, the plurality of MSCs are cultivated on culture vessel surface having a coating that promotes cell adhesion for at least one passage prior to the culturing on the modified surfaced without a coating. In some of the methods provided, the plurality of MSCs are cultivated on culture vessel surface having a coating that promotes cell adhesion for up to five passages prior to the culturing on the modified surfaced without a coating. In some of the methods provided, the plurality of MSCs are cultivated on culture vessel surface having a coating that promotes cell adhesion for one, two, three, four, or five passages prior to the culturing on the modified surfaced without a coating.

Modified surfaces suitable for use as substrates in the methods and compositions of the present disclosure may be surfaces that have been modified to contain from at least about 0.9% N, a sum of O and N of greater than or equal to 19.0% and a contact angle of at least about 14 degrees. In some embodiments, such a surface may be a vessel surface. Alternatively, the surface may be a 3-dimensional matrix, such as, for example, a porous scaffold to which the cells can attach. The modified surfaces may be comprised of any material that is capable of providing support onto which mesenchymal stem cells may attach and expand in culture medium, for example in serum-free culture medium. For example, the modified surface may be comprised of polystyrene, cyclic olefin polymer, cyclic olefin copolymer, polyolefin, polycarbonate, polymethyl methacrylate, styrene acrylonitrile copolymer, and mixtures and copolymers therefrom.

The modified surfaces for use in the methods and compositions provided herein may be prepared by treating injection molded items (such as, without limitation, culture vessels, dishes, plates, flasks, bottles) using a vacuum plasma treatment, such as made by radio-frequency or microwave plasma treatment. Exemplary surface treatments and modifications suitable for use in the present disclosure include, but are not limited to, those described in U.S. Pat. No. 10,066,203, which is incorporated by reference herein for such teachings.

The plasma treatment may be carried out in a metal vacuum chamber with one electrode or with multiple electrodes inside the chamber and electrically isolated from the inside of chamber (Tantec A/S, Denmark). The metal walls serve as counter electrode (ground). One or more self-tuning generators generates the electrical field giving sufficient energy to generate plasma in the entire chamber. In an exemplary surface treatment, an item to be treated is placed on a shelf in the chamber and the chamber is closed and evacuated to a desired pressure. At this pressure the valve to the vacuum pump is closed and the generator engaged. The generator is set to generate a desired power output and the pressure is again controlled at a desired value for a period of time, for example about 5 to 120 seconds. The gas inlet valve (air) is then opened, and the pressure in the chamber returned to atmospheric level.

The vacuum plasma process is carried out in a chamber at a pressure of about 0.01-3.0 mbar, depending on the exact application. High voltage electrodes are mounted in the chamber and though these electrodes a high voltage high frequency power discharge (from 9 kHz to 1 GHz in the RF area) is imposed and generates the plasma discharge, which is seen as a light purple light inside the vacuum chamber. The discharge and its power is defined by the size and number of electrodes (for example, above about 2900 W) along with the size of the power supply. The plasma discharge serves the purpose of changing the molecule structure on the substrate surface and so imposing new properties to the substrate. The treatment time is adjustable and generally in the range from 5 to 120 seconds, typically in the range from 30-90 seconds. Various treatment gasses may be used depending on the desired properties for the treated substrate including, for example, normal air and filtered, compressed air.

In other embodiments, microwave plasma treatment may be carried out in a quartz vacuum chamber (for example, Model 300-E or Model 440 from Technics Plasma GmbH, Germany). The energy to generate the plasma is supplied by a 2.43 GHz microwave generator outside the chamber. In an exemplary surface treatment, an item to be treated is placed on a glass plate inside the chamber. The chamber is closed and evacuated to a desired pressure, for example between 0.3 and 0.5 mbar. The valve to the vacuum pump is kept open, and the pressure is maintained at the desired value by adjusting gas (air or oxygen) flow with the gas inlet valve. The microwave generator is then engaged. The generator is set to generate an output of, for example, 500 or 600 W and the plasma energized for about 6 to 60 seconds. The pump valve is then closed, and the air inlet valve opened, in order to bring the pressure in the chamber to atmospheric level.

Culture plate surfaces were modified with a plasma treatment as described and used in the examples provided herein. The plasma surface modified plates were packed in plastic bags, then sterilized by gamma irradiation (25 kGy), and stored at room temperature until used in cell culture experiments or surface characterization analysis. Vessels with the Nunclon Delta™ surface were used in cell culture experiments as a control surface. The Nunclon Delta™ surface also underwent surface characterization analysis.

The elemental composition of the modified surfaces of the methods and composition provided herein were analyzed by X-Ray Photoelectron Spectroscopy (XPS). XPS, also known as Electron Spectroscopy for Chemical Analysis (ESCA), is used as a method to determine what elements or atoms are present in the surface of a solid substrate (all elements in concentrations greater than 0.1 atomic percent can be detected, except hydrogen and helium), and to determine the bonding environment of such elements or atoms. As an example, an XPS analysis of a polystyrene (contains only carbon and hydrogen) solid sample would typically give greater than 97% carbon, less than 3% oxygen, and 0% nitrogen (hydrogen is not detected; different levels of oxygen may be detected due to oxidation of the polystyrene chains at the surface, for example, as a result of sterilization by irradiation) (Brevig et al., Biomaterials 26:3039-3053, 2005; Shen and Horbett, J. Biomed. Mater. Res. 57:336-345, 2001).

As determined by XPS analysis, the modified surfaces provided herein contain carbon, oxygen and nitrogen (hydrogen is not detected in XPS) in the surface. In some embodiments, the modified surfaces for use in the methods and compositions provided herein contained at least 0.9% N and a sum of O and N of at least 19%. In some embodiments, the modified surfaces contained a % N in the range of about 0.9% to about 3.2% and a sum of N and O in the range of about 19 to about 35%. In comparison, the elemental composition of the Nunclon Delta™ surface is 0.6% N and a sum of O and N of 15.3% (see, for example, U.S. Pat. No. 10,066,203).

XPS analysis of the surfaces provided herein may be performed, for example, using a Model 5400 X-Ray Photoelectron Spectrometer (Physical Electronics, Inc.). The analysis may include, without limitation, presenting surface samples to the x-ray source by cutting sections from plates with the modified surface, mounting them onto a stainless steel sample holder, and irradiating with A1 kα radiation (1486 eV). The analysis may be performed with an angle of 45° between the sample and analyzer and the spectra curve fit using commercial Matlab routines for data processing and software provided by the instrument manufacturer. For example, the outermost two to five nanometers in depth in a region of about one millimeter in diameter from the surface treated part of the plates was analyzed in each of two plates per surface.

The roughness of the modified surfaces of the methods and compositions provided may be analyzed by Atomic Force Microscopy (AFM). For example, AFM may be used to acquire the profiles of the modified surface with an NX20 Atomic Force Microscope (Park Systems) in intermediate contact mode or non-contact mode using an AFM tip with a nominal radius of ≤20 nm. Other methods for analyzing roughness using AFM are known and described, for example, in U.S. Pat. No. 10,066,203.

The wettability of the modified surfaces of the methods and compositions provided may be analyzed by measuring the contact angle. For example, contact angle measurement by the static sessile drop method provides information on the interaction between the surface of a solid substrate and a liquid. The contact angle describes the shape of a liquid drop resting on the surface of the solid substrate, and is the angle of contact of the liquid on the surface of the solid substrate, measured within the liquid at the contact line where liquid, solid, and gas meet. A surface with a water contact angle larger than 90° is termed hydrophobic, and a surface with water contact angle less than 90° is termed hydrophilic. On extremely hydrophilic surfaces, that is, surfaces that have a high affinity for water, a water droplet will completely spread (an effective contact angle of 0°).

The water contact angle of the surfaces provided herein may be determined by top-view measurements or by side-view measurements. For example, contact angle measurements may be done using the static sessile drop method and a PG-X measuring Head from FIBRO Systems AB, Sweden [goniometer consisting of video camera and computer software (v. 3.1)]. The tangent leaning method may be used for calculation of the contact angles, according to the manufacturer instructions. Measurements on Nunclon Delta™ surface were performed under the same experimental conditions and have been described, for example, in U.S. Pat. No. 10,066,203. The modified surfaces used herein were more hydrophilic (lower water contact angles) than Nunclon Delta™ surface. The modified surfaces for use in the methods and compositions provided herein contain a static sessile contact angle of at least about 14 degrees. For example, the modified surfaces contain a static sessile contact angle of about 14 degrees to about 55 degrees. In comparison, the static sessile contact angle the Nunclon Delta™ surface is about 63.1 degrees (see, for example, U.S. Pat. No. 10,066,203).

The negative charge density of the modified surfaces of the methods and compositions provided may be analyzed. For example, the negative charge density of the surfaces may be analyzed by measuring the reactivity of the surface with crystal violet. Crystal violet carries a positive charge, which enables it to bind to negatively charged molecules and parts of molecules, for example, negatively charged functional groups present on a polymer surface. A surface with a high crystal violet reactivity has a higher density of negative charges than a surface with a low crystal violet reactivity, given that the surfaces have the same roughness and thus area. Methods for analysis of negative charge density using crystal violet are known and described, for example, in U.S. Pat. No. 10,066,203.

A mesenchymal stem cell (MSC) is a progenitor cell having the capacity to differentiate into different types of cells such as, but not limited to, adipocytes, chondrocytes, osteoblasts, myocytes, tenocytes, cardiomyocytes and cardiac tissue, hepatocytes, keratocytes, neurocytes and all varieties and derivatives of neuroectodermal cells, and other endothelial and epithelial cells. These cells may be defined phenotypically by gene or protein expression. MSCs have been characterized to express (and thus be positive for) one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-bIR, TGF-bIIR, HLA-A, B, C, SSEA-3, SSEA-4, D7 and PD-L1. MSCs have also been characterized as not expressing (and thus being negative for) one or more of CD3, CD5, CD6, CD9, CD10, CD11a, CD14, CD15, CD18, CD21, CD25, CD31, CD34, CD36, CD38, CD45, CD49d, CD50, CD62E, L, S, CD80, CD86, CD95, CD117, CD133, SSEA-1, and ABO. Thus, MSCs may be characterized phenotypically and/or functionally according to their differentiative potential.

Mesenchymal stem cells used in the methods and compositions provided herein may be derived from a number of sources including but not limited to bone marrow, blood, periosteum, adipose tissue, dermis, umbilical cord blood and/or matrix (e.g., Wharton's jelly), placenta, synovial fluid, synovial membrane, deciduous teeth and muscle tissue. Methods for harvesting and isolating mesenchymal stem cells are known and described in the art. Generally, the process for MSC harvesting, isolation and/or identification involves isolation of various tissues, digestion to obtain cells, and culture for a period of time (e.g., 3 to 5 days), followed by discarding non-adherent cells and continuous culture of adherent cells to the desired passage. MSCs can be identified by a variety of markers expressed within the cell or on the cell surface as described herein.

The mesenchymal stem cells contemplated for use in the methods provided herein may be derived from the same subject to be treated (and therefore would be referred to as autologous to the subject) or they may be derived from a different subject preferably of the same species (and therefore would be referred to as allogeneic to the subject).

As used herein, it is to be understood that aspects and embodiments of the disclosure relate to cells as well as cell populations, unless otherwise indicated. Thus, where a cell is recited, it is to be understood that a cell population is also contemplated unless otherwise indicated.

As used herein, an isolated mesenchymal stem cell is a mesenchymal stem cell that has been physically separated from its natural environment, including physical separation from one or more components of its natural environment. Thus, an isolated cell or cell population embraces a cell or a cell population that has been manipulated in vitro or ex vivo. As an example, isolated mesenchymal stem cells may be mesenchymal stem cells that have been physically separated from at least 50%, preferably at least 60%, more preferably at least 70%, and even more preferably a least 80% of the cells in the tissue from which the mesenchymal stem cells are harvested. In some instances, the mesenchymal stem cells are present in a population that is at least 20%, at least 30%, least 40%, at least 50%, at least 60%, at least 70%, at least 80%, at least 85%, at least 90%, at least 95%, at least 96%, at least 97%, at least 98%, at least 99%, or 100% mesenchymal stem cells as phenotypically and/or functionally defined herein. Preferably the ratio of mesenchymal stem cells to other cells is increased in the expanded cell population as compared to the starting population of cells.

Mesenchymal stem cells can be isolated using methods known in the art, e.g., from bone marrow mononuclear cells, umbilical cord blood, adipose tissue, placental tissue, synovium, based on their adherence to tissue culture plastic. For example, mesenchymal stem cells can be isolated from commercially available bone marrow aspirates, adipose tissue samples, and Wharton's jelly samples. Enrichment of mesenchymal stem cells within a population of cells can be achieved using methods known in the art including but not limited to FACS. See, for example, Han et al. (2019) Cells 8:886 and U.S. Pat. Nos. 9,901,600 and 9,085,755.

Mesenchymal stem cells from human and other animal sources are also commercially available from, for example, Thermo Fisher Scientific, Inc., Accegen Biotechnology and PromoCell GmbH.

The isolation and cultivation of the population of mesenchymal stem cells can be carried out under standard condition for the cultivation of mammalian cells. Typically, the methods provided herein of expanding a population of mesenchymal stem cells is typically carried out at conditions (temperature, atmosphere) that are normally used for cultivation of cells of the species of which the cells are derived. For example, human mesenchymal stem cells are usually cultivated at 37° C. in air atmosphere with 5% CO₂. In this context, it is noted that the in the provided methods the mesenchymal stem cells may be derived of any mammalian species, such as mouse, rat, guinea pig, rabbit, goat, horse, dog, cat, sheep, monkey or human, with mesenchymal stem cells of human origin being preferred in one embodiment.

Commercially available media may be used for the growth, culture and maintenance of mesenchymal stem cells. In embodiments, the growth medium includes a basal medium and a supplement. In embodiments, the complete growth medium (basal medium with all supplements added) is serum-free. In embodiments, the complete growth medium is serum-free and animal-origin-free. In embodiments, the complete growth medium is xeno-free and serum-free.

In embodiments, the basal medium is selected from StemPro™ MSC SFM Basal Medium (GIBCO); StemPro™ MSC SFM XenoFree Basal Medium (GIBCO); MesenPRO RS™ Basal Medium (GIBCO); Dulbecco's Modified Eagle Media (DMEM); DMEM low glucose; DMEM high glucose; DMEM/F12; DMEM/F12 phenol red-free, no HEPES; DMEM/F12 with phenol red, no HEPES; DMEM/F12 with HEPES and phenol red; DMEM/F12 with HEPES, no phenol red; DMEM/F12 with or without GLUTAMAX™ (Thermo Fisher Scientific); Advanced DMEM/F12; MSC NutriStem® XF Medium (Satorius); RPMI 1640 Medium, Minimum Essential Medium α (MEMα), Iscove's Modified Dulbecco's Medium (IMDM), and Basal Medium Eagle (BME).

Components in such media that are useful for the growth, culture and maintenance of mesenchymal stem cells include but are not limited to amino acids, vitamins, a carbon source (natural and non-natural), salts, sugars, plant derived hydrolysates, sodium pyruvate, surfactants, ammonia, lipids, hormones or growth factors, buffers, non-natural amino acids, sugar precursors, indicators, nucleosides and/or nucleotides, butyrate or organics, DMSO, animal derived products, gene inducers, non-natural sugars, regulators of intracellular pH, betaine or osmoprotectant, trace elements, minerals, non-natural vitamins. In some embodiments, the amino acid is a stable analog of an amino acid (e.g., GlutaMAX™ Supplement and GlutaMAX™_I CTS™ Supplement, available from Thermo Fisher Scientific). In some embodiments, the medium and/or supplement comprises an amino acid derivative, e.g., N-acetyl-L-cysteine. Additional components that can be used to supplement a commercially available tissue culture medium for certain steps or stages of the provided methods include, for example, animal serum (e.g., fetal bovine serum (FBS), fetal calf serum (FCS), horse serum (HS)), antibiotics (e.g., including but not limited to, penicillin, streptomycin, neomycin sulfate, amphotericin B, blasticidin, chloramphenicol, amoxicillin, bacitracin, bleomycin, cephalosporin, chlortetracycline, zeocin, and puromycin), and glutamine (e.g., L-glutamine). Mesenchymal stem cell survival and growth also depends on the maintenance of an appropriate aerobic environment, pH, and temperature. Mesenchymal stem cells can be maintained using methods known in the art. (See for example Pittenger et al., Science, 284:143-147 (1999).)

In an aspect of the provided methods, MSCs are expanded in culture while maintaining pluripotency. For example, MSCs are expanded in culture while maintaining the trilineage mesoderm differentiation potential of human MSCs. Changes in pluripotency of the cells with time can be determined by detecting changes in the levels of expression of markers associated with MSC pluripotency, such as those markers described herein. In the provided methods, maintenance of MSC pluripotency is detected by expression cell surface markers CD90, CD73, CD105 and CD44 and a lack of expression of significant amounts of CD34, CD45, CD14 and CD79. Alternatively, changes in pluripotency can be monitored by detecting changes in the levels of expression of markers associated with differentiation or markers associated with another cell type.

In another aspect, MSCs are expanded in culture in accordance with the methods provided and then treated in a manner that promotes their differentiation into another cell type. The other cell type may be a cell expressing markers characteristic of the chrondrocyte lineage. Alternatively, the cell type may be a cell expressing markers characteristic of the osteoblast lineage. Alternatively, the cell type may be a cell expressing markers characteristic of the adipocyte lineage. Alternatively, the cell type may be a cell expressing markers characteristic of the myoblast lineage. Alternatively, the cell type may be a cell expressing markers characteristic of the fibroblast lineage. Alternatively, the cell type may be a cell expressing markers characteristic of the tenoblast lineage.

Mesenchymal stem cells treated in accordance with the methods provided herein may be differentiated into a variety of other cell types by any suitable method in the art. For example, MSCs expanded in accordance with the methods provided herein may be differentiated into osteocytes, adipocytes, chondrocytes, skeletal myocytes, cardiac myocytes, smooth muscle myocytes, stromal fibroblasts, tenocytes, hepatocytes, and neuronal cells.

Commercially available MSC differentiation kits and growth factors may be used for the differentiation of expanded population of mesenchymal stem cells to a variety of cell types. Examples of such kits include Gibco™ StemPro™ Adipogenesis Differentiation kit, Gibco™ StemPro™ Osteogenesis Differentiation kit, and Gibco™ StemPro™ Chondrogenesis Differentiation kit (all from Thermo Fisher Scientific).

In one aspect, the present disclosure relates to a method of treating a subject in need of a therapy, the method including: (i) obtaining mesenchymal stem cells (MSCs); (ii) expanding the MSCs in a serum-free growth medium on a surface lacking a coating that promotes cell adhesion and lacking a feeder cell layer; and (iii) transferring the expanded population of MSCs to the subject, thereby treating the subject.

Accordingly, the present disclosure provides a method of treating a subject in need of a therapy, the method including: (i) obtaining mesenchymal stem cells (MSCs); (ii) expanding the MSCs in a serum-free growth medium on a modified surface containing at least about 0.9% N, a sum of O and N of greater than or equal to 19.0% and a contact angle of at least about 14 degrees, and lacking a coating that promotes cell adhesion and lacking a feeder cell layer; and (iii) transferring the expanded population of MSCs to the subject, thereby treating the subject.

MSCs expanded in vitro or ex vivo according to any aspect of this disclosure may be used as a medicament. For example, a population of MSCs expanded in a serum-free growth medium on a surface, where the surface lacks a coating that promotes cell adhesion and lacks a feeder cell layer, may be used as a medicament to treat a subject. For another example, a population of MSCs expanded in a serum-free growth medium on a modified surface, where the surface contains at least about 0.9% N, a sum of O and N of greater than or equal to 19.0% and a contact angle of at least about 14 degrees, and lacks a coating that promotes cell adhesion and lacks a feeder cell layer, may be used as a medicament to treat a subject. The MSCs may be derived from the same subject that is to be treated and/or may be genetically modified prior to transferring the MSCs to the subject. The MSCs may be derived from individuals other than the subject that is to be treated and used as allogeneic MSC transplants in treating a subject. Expanded MSCs may be used to treat a wide variety of conditions including but not limited to immune system disorders, inflammatory disease or disorders, ischemia-related disorders or conditions, and diseases or conditions that would benefit from tissue sparing and/or regeneration. For example, expanded MSCs may be used to treat conditions including but not limited to graft-versus-host disease, Crohn's disease, type I diabetes mellitus, myocardial infarction, chronic obstructive pulmonary disease, conditions associated with skeletal dysplasia, metachromatic leukodystrophy, Hurler syndrome, liver cirrhosis, spinal cord injury, critical-limb ischemia, and/or renal failure in a subject. The subject may be human.

In another aspect, the present disclosure relates to a method for improving engraftment potential of a population of mesenchymal stem cells (MSCs), the method including: (a) obtaining a population of MSCs; and (b) expanding the population of MSCs in a serum-free growth medium on a surface lacking a coating that promotes cell adhesion and lacking a feeder cell layer, thereby improving engraftment potential of the population of MSCs.

Accordingly, the present disclosure provides a method for improving engraftment potential of a population of mesenchymal stem cells (MSCs), the method including: (a) obtaining a population of MSCs; and (b) expanding the population of MSCs in a serum-free growth medium on a modified surface containing at least about 0.9% N, a sum of O and N of greater than or equal to 19.0% and a contact angle of at least about 14 degrees, and lacking a coating that promotes cell adhesion and lacking a feeder cell layer, thereby improving engraftment potential of the population of MSCs.

In an aspect, the present disclosure relates to a method for editing a genome in a mesenchymal stem cell (MSC), the method including: (a) obtaining a MSC that was expanded using the expansion methods provided herein; and (b) editing the genome of the MSC. In embodiments, the genome is edited using one or more genome editing reagents selected from a zinc-finger nuclease (ZFN), transcription activator-like effector nuclease (TALEN), meganuclease, and a clustered regularly interspaced short palindromic repeat (CRISPR) associated protein, and (c) optionally expanding the edited cell in MSC on the modified surface and serum-free media as described herein for a period of time (e.g., 1 day, 2 days 3 days, 4 days, 5 days, 6 days, 1 week, 2 weeks, or longer, or any amount of time in between).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

The modified surfaces provided herein were assessed in culturing and expanding MSC populations in serum-free conditions without a surface coating of an ECM-based matrix, an ECM protein or a synthetic matrix for cell adherence and expansion. Expansion of MSCs on an exemplary modified surface provided herein (Surface A) which was uncoated was compared to expansion of the cells on the control surfaces of Nunclon™ Delta plates and Surface A plates coated with a synthetic matrix for cell adherence and expansion.

Cryopreserved mesenchymal stem cells (MSC) were initially recovered and maintained in serum-free media on a vessel surface coated with CELLstart™ CTS™ Substrate (Thermo Fisher Scientific). For this, CELLstart™ CTS™ Substrate was diluted 1:100 in Dulbecco's Phosphate Buffered Saline (DPBS) CTS™ with Ca²+ and Mg²+. The coating solution was added to Nunclon™ Delta cell culture treated vessels. They were placed with the CELLstart substrate solution in the incubator at 37° C. in a humidified atmosphere of 5% CO₂ for 60 minutes. After incubation, vessels were removed from the incubator and temporarily placed in a laminar flow hood until use. Immediately before use, all CELLstart™ CTS™ substrate was removed and replaced with complete medium.

Cell culture medium used for growth was Gibco™ StemPro™ MSC SFM Basal Medium supplemented with StemPro™ MSC SFM XenoFree Supplement and Gibco™ GlutaMAX™-I CTS™ at a 1× concentration to form complete medium. Human MSCs derived from three different sources were used: bone marrow derived, adipose derived and Wharton's Jelly derived. To recover cryopreserved human MSCs, a frozen vial of cells was rapidly thawed in a 37° C. water bath. The contents of the cryovial were put in a 50-mL conical tube. Complete, pre-warmed StemPro™ MSC SFM XenoFree medium was added to the tube to a volume of 10 mL dropwise while gently swirling the tube.

The cells were then centrifuged at 150×g for 5 minutes at room temperature. The cell pellet was resuspended in complete medium, and the suspension was counted using trypan blue exclusion to assess % viability. Cells were seeded into cell culture vessels coated with CELLstart™ CTS™ substrate at a density of 5×10³ cells/cm². Cells were incubated at 37° C. with a humidified atmosphere of 5% CO₂ in air. Complete StemPro™ MSC SFM XenoFree medium was replaced every 2-3 days.

Once the cell monolayer reached 60-90% confluency, the cells were subcultured onto a new vessel surface. For this, spent medium was removed from the culture vessel containing the cell monolayer and the monolayer was carefully rinsed with 10 mL of DPBS CTS™ without Ca+² and Mg+². TrypLE™ CTS™ (Thermo Fisher Scientific) reagent was added to the vessel at a volume sufficient to cover the bottom of the cell culture vessel and then distributed evenly over the cell monolayer. The cells were incubated at 37° C. with a humidified atmosphere of 4% CO₂ in air for 5 minutes or until the cells dislodged.

Complete StemPro™ MSC SFM XenoFree medium was pre-warmed to 37° C. before use. The cell suspension was collected into a 50 mL conical tube containing complete medium and centrifuged at 150×g for 5 minutes at room temperature. Cells were counted using trypan blue exclusion to assess % viability.

At this time, a vessel having Surface A, the modified surface provided herein, was prepared by adding only StemPro™ MSC SFM XenoFree complete medium. This vessel was not coated with any extra-cellular matrix or other cell adhesive coating. A new Nunclon Delta cell culture vessel and a new Surface A cell culture vessel were coated with CELLstart™ CTS™ Substrate as described above. The CELLstart™ CTS™ substrate solution was the removed from each coated vessel and add StemPro™ MSC SFM XenoFree complete medium was added.

The cells were then seeded into the one uncoated vessel and the two coated control vessels. They were evenly distributed into the vessels at a density of 5×10³ cells/cm². The vessels were incubated at 37° C. with a humidified atmosphere of 5% CO₂. The culture medium was replaced every 2-3 days. Cells were subcultured at least two more times before differentiation.

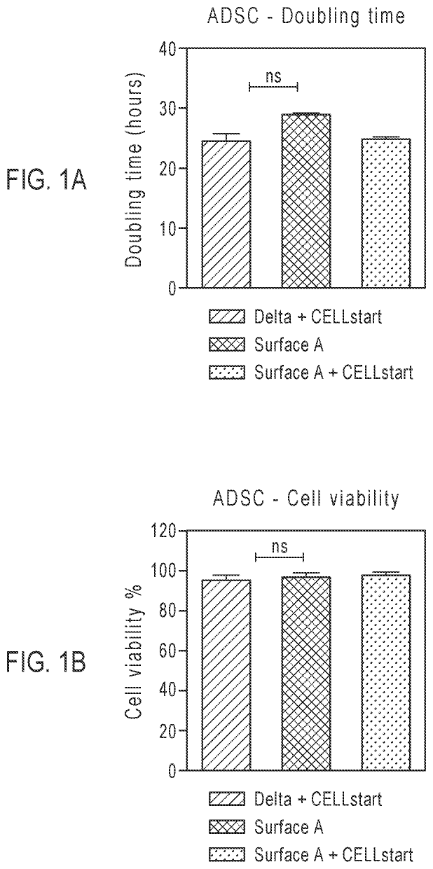

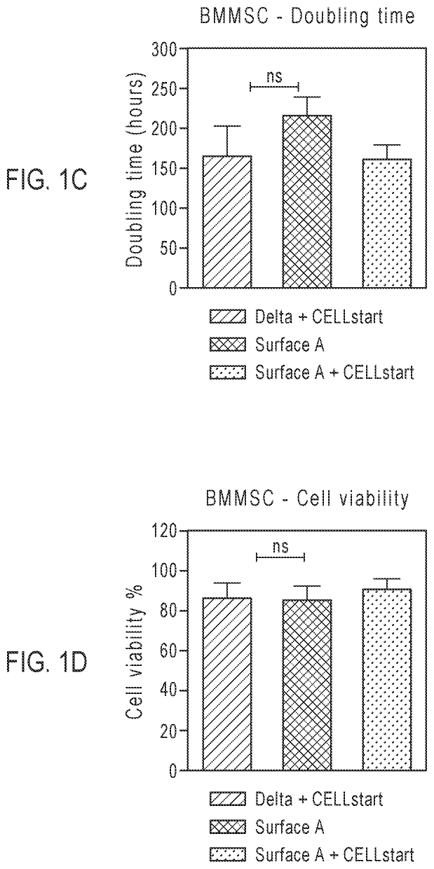

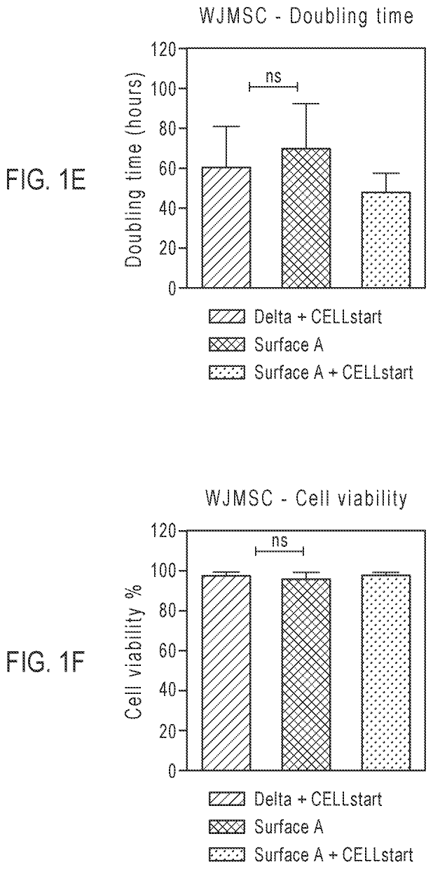

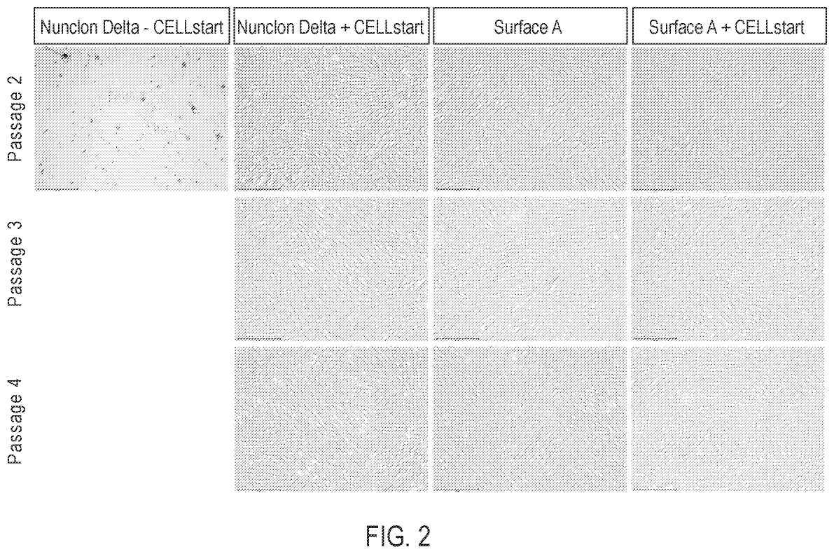

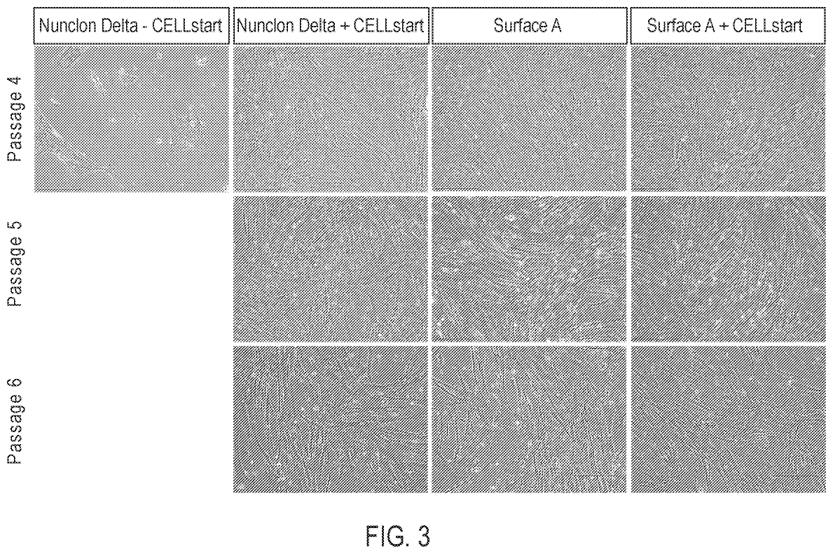

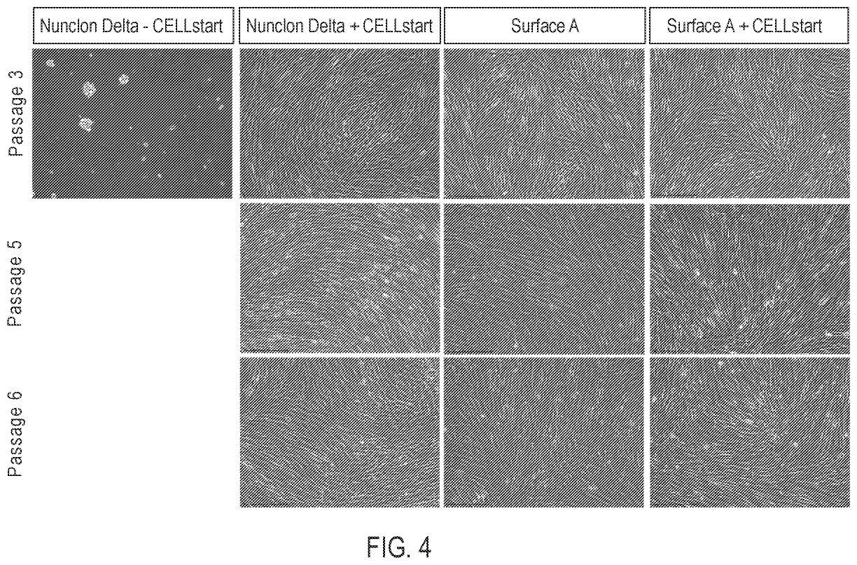

Human mesenchymal stem cells (hMSCs) derived from bone, adipose, and Wharton's Jelly proliferate on the uncoated Surface A in the absence of serum (see FIGS. 1A-1F). None of the MSC cell types tested attached well for growth or survived multiple cell passages when cultured on the Nunclon Delta surface without a coating and without serum in the media (FIGS. 2-4). In serum-free medium, there is no significant difference in cell growth or viability when MSCs are cultured on the uncoated Surface A as compared to the CELLstart-coated Delta surface or CELLstart-coated Surface A (FIGS. 1A-1F). In the serum-free medium, cell morphology remains similar on the uncoated Surface A and coated Delta surface despite the Surface A culture not having a matrix coating (FIGS. 2-4). As shown in FIG. 2, adipose derived MSCs in serum-free media grown on the coated Delta surface and the uncoated Surface A have a normal cell morphology over 4 passages. As shown in FIG. 3, bone marrow derived MSCs in serum-free media grown on the coated Delta surface and the uncoated Surface A have a normal cell morphology over 6 passages. As shown in FIG. 4, Wharton Jelly-derived MSCs in serum-free media grown on the coated Delta surface and the uncoated Surface A have a normal cell morphology over 6 passages.

After passaging the cells on the coated Delta surface or the uncoated Surface A, a portion of the populations were processed for flow cytometry analysis and a portion were differentiated into chondrocytes, osteocytes, and adipocytes.

Cell Surface Analysis. To assess the phenotype of the mesenchymal stem cells cultured in serum free media on the Delta surface and the Surface A surface, cells were suspended in eBioscience™ Flow Cytometry Staining Buffer (Invitrogen, Thermo Fisher Scientific) at a concentration is 2.5 million cells per mL. One hundred microliters of the cell suspension were then distributed into a 96 well round bottom plate. To reduce non-specific binding, human Fc block was added and incubated at 4° C. After a 20-minute incubation, the recommended dilutions of antibodies and the corresponding isotype control were added to the wells. Cells were incubated with the antibodies for the manufacturer's recommended amount of time. Cells were then washed in Flow Cytometry Staining Buffer and re-suspended in DPBS. Secondary antibodies were diluted according to manufacturer's recommendations and added where necessary. Cells were washed and stained with a live/dead stain. Cells were washed again in DPBS and then analyzed on Attune NxT flow cytometer.

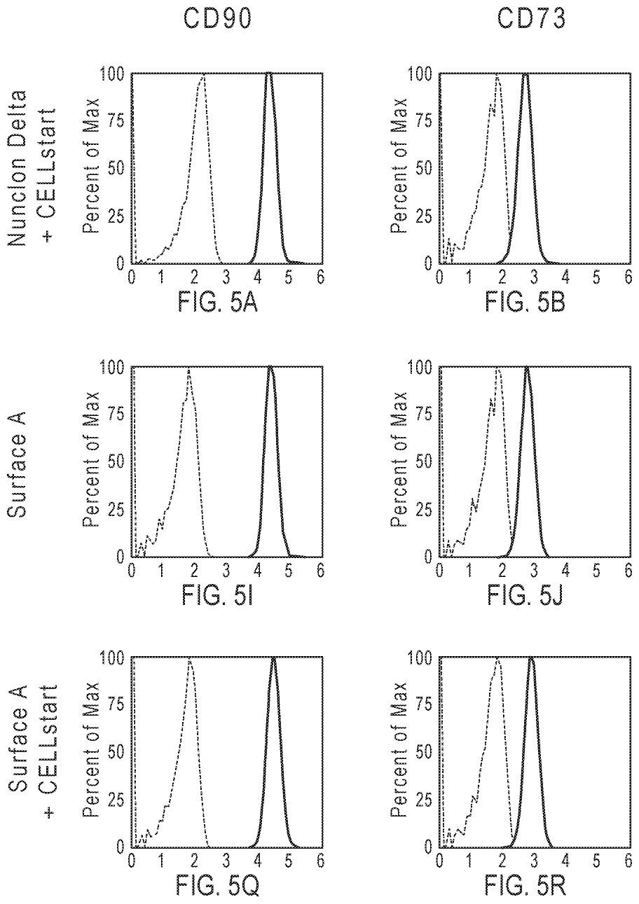

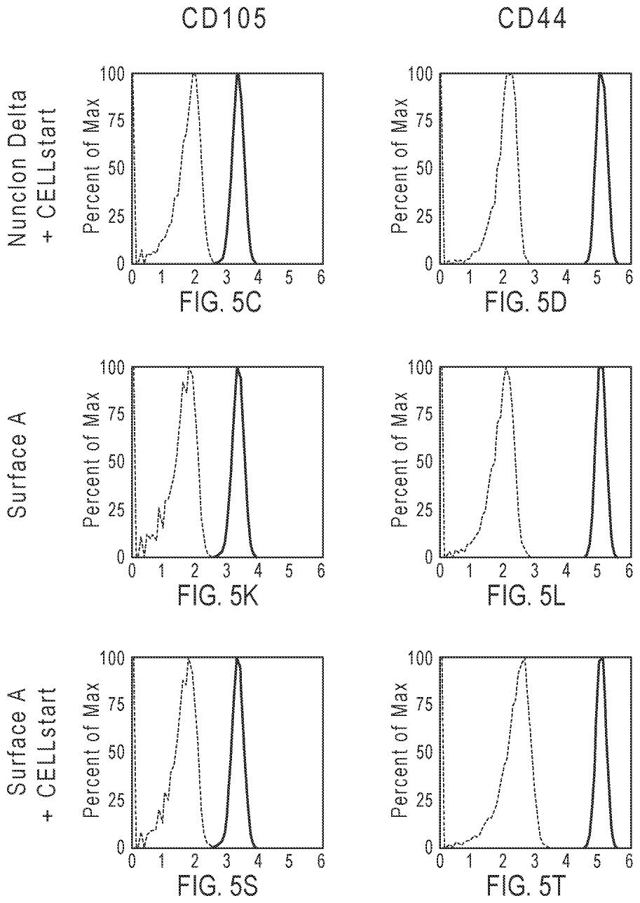

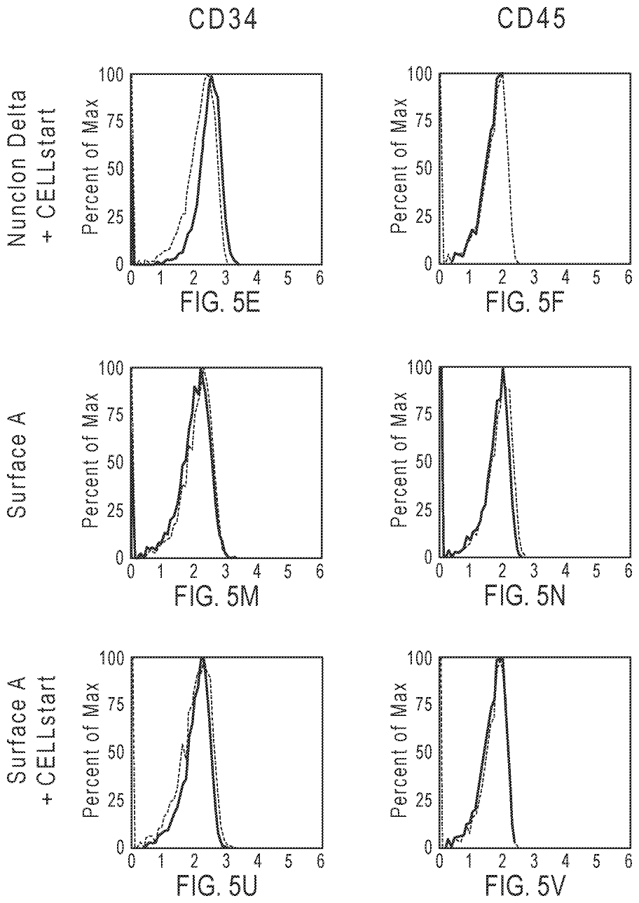

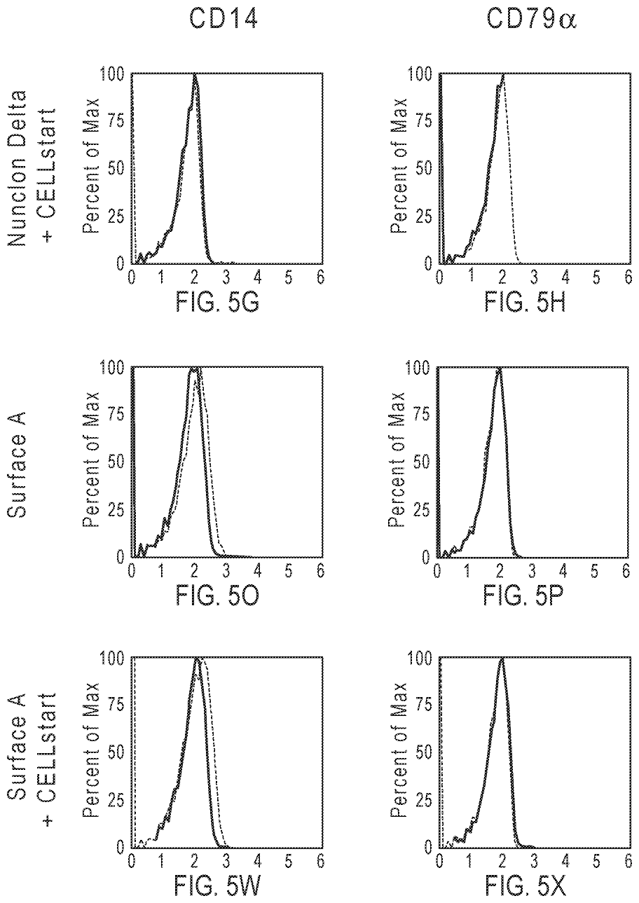

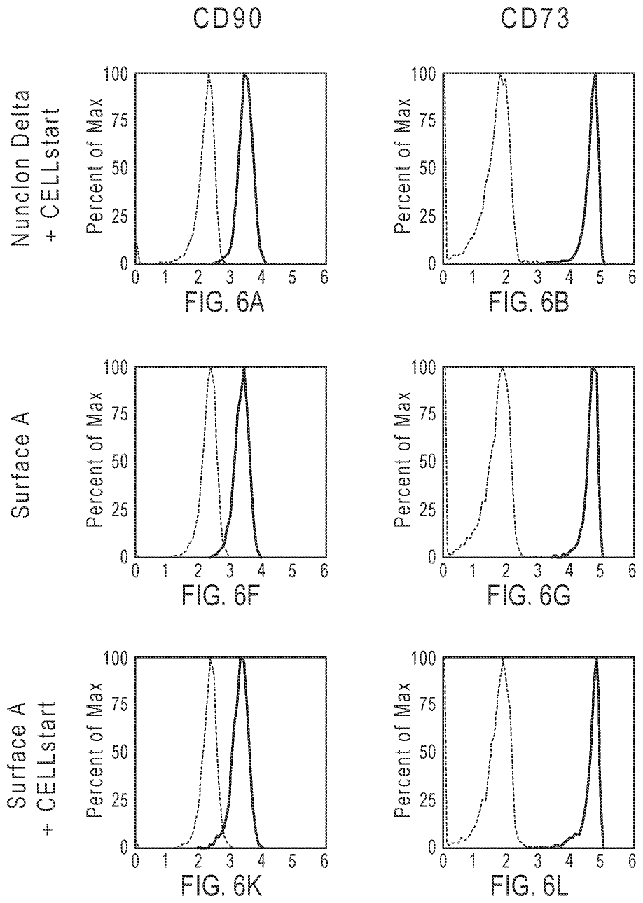

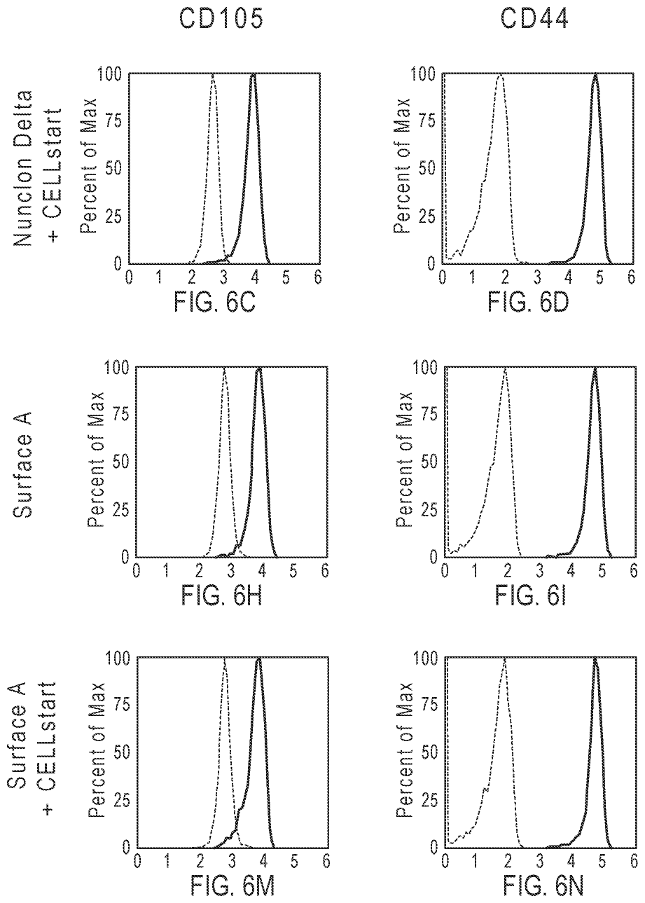

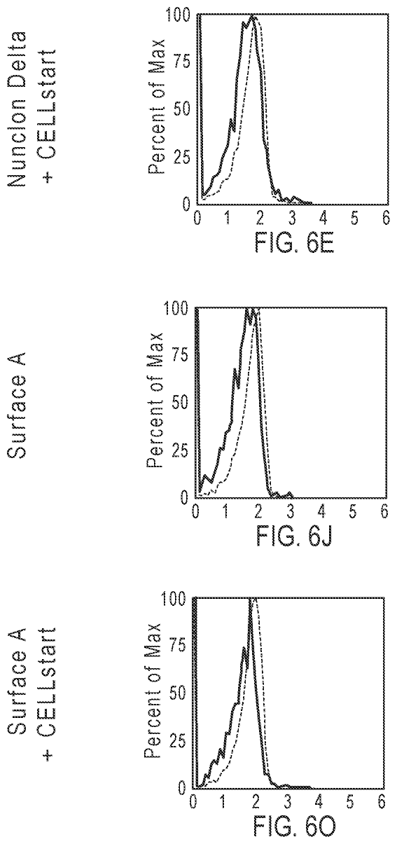

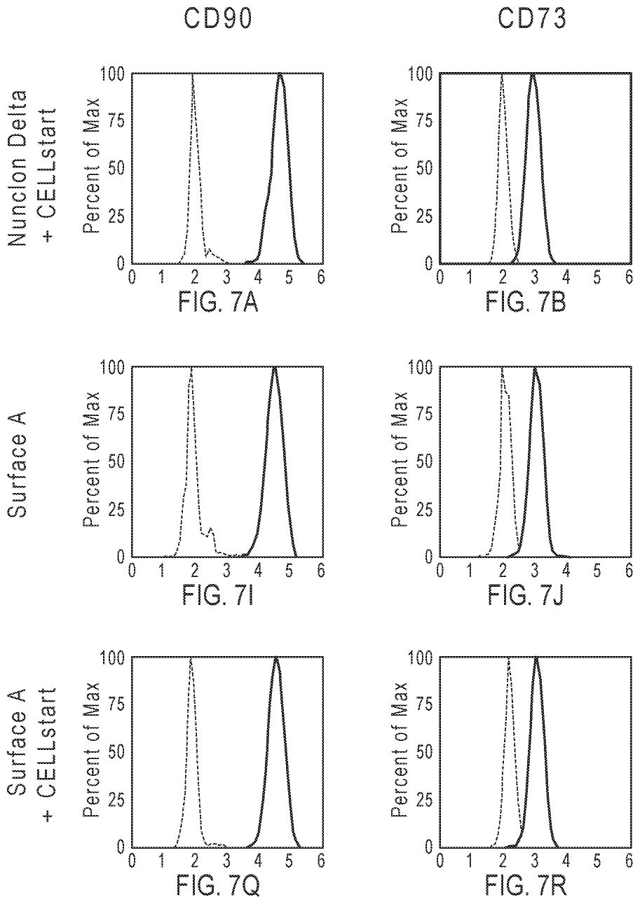

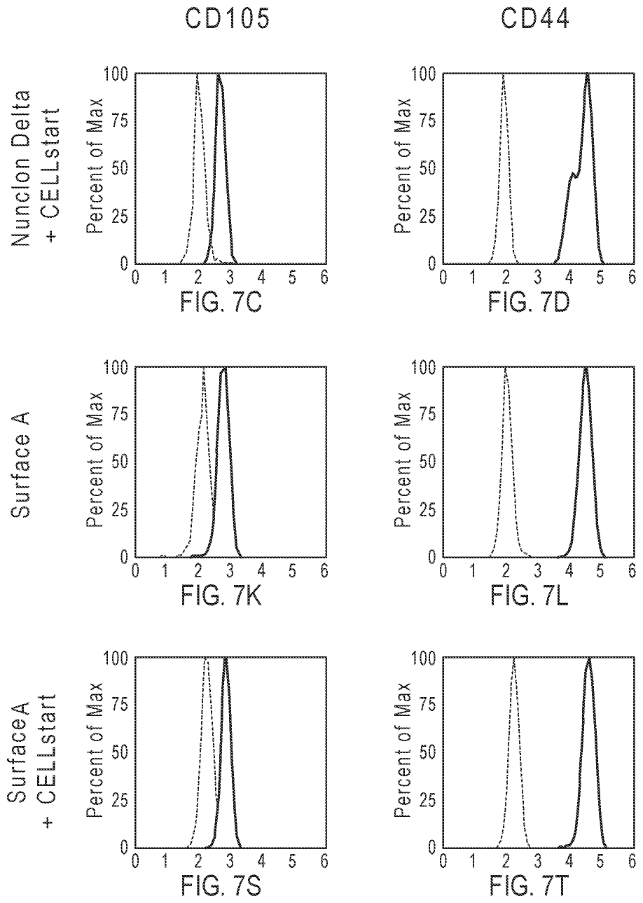

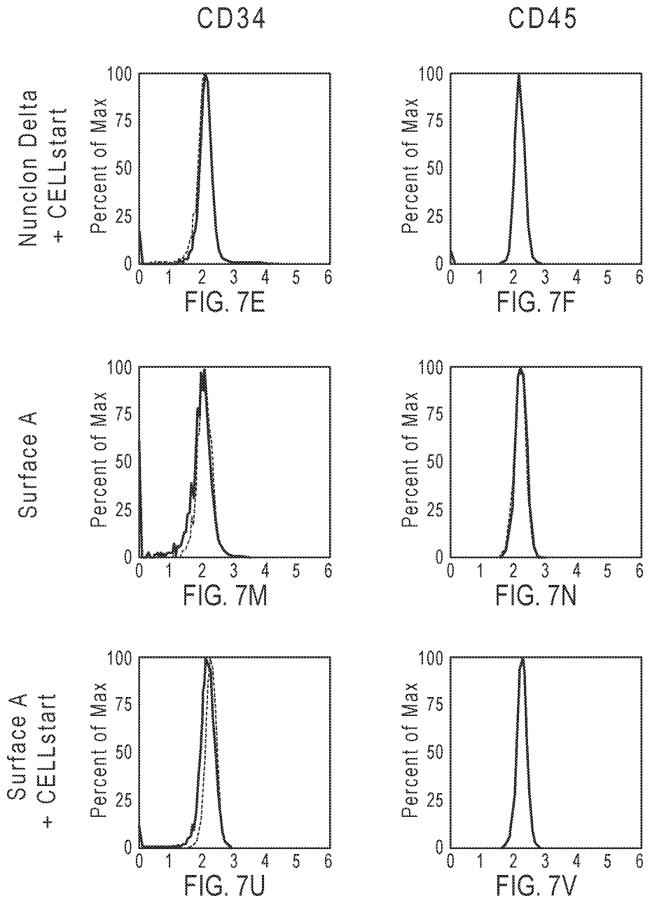

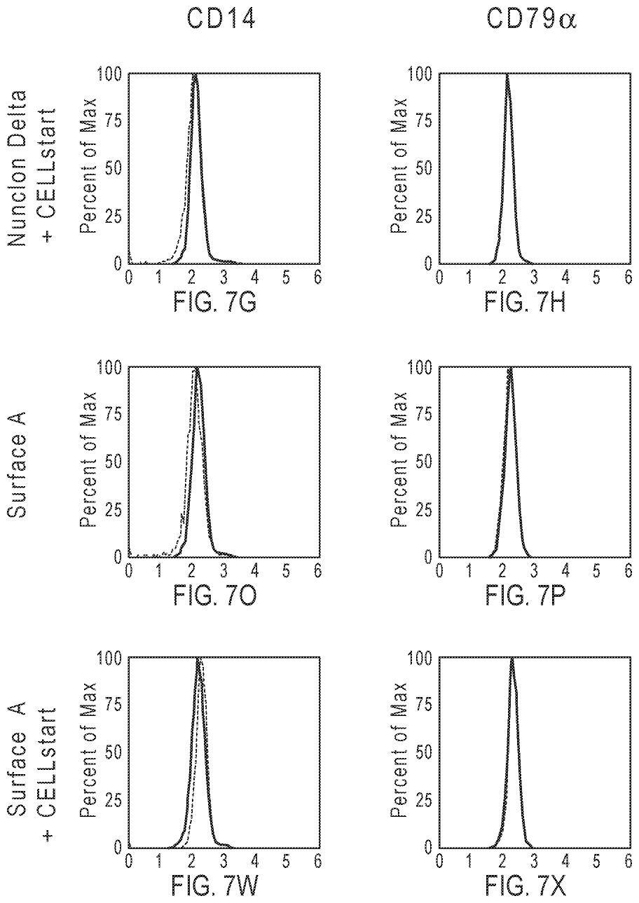

The flow cytometry analysis demonstrated that the expanded MSCs maintained their pluripotency as indicated by presence of cell surface markers CD90, CD73, CD105, and CD44 (FIGS. 5-7). The cells did not display significant amounts of CD34, CD45, CD14, and CD79 further supporting the conclusion that they were pluripotent. As shown in FIGS. 5A-5X, adipose derived hMSCs retained their pluripotency markers when cultured on Surface A in absence of any extracellular matrix coating and serum. Flow cytometry analysis shows that the phenotype of these cells on the uncoated Surface A is equivalent to that on the coated surfaces. As shown in FIGS. 6A-6O, bone marrow derived hMSCs retained their pluripotency markers when cultured on Surface A in absence of any extracellular matrix coating and serum. Flow cytometry analysis shows that the phenotype of these cells on the uncoated Surface A is equivalent to that on the coated surfaces. As shown in FIGS. 7A-7X, Warton's Jelly derived hMSCs retained their pluripotency markers when cultured on Surface A in absence of any extracellular matrix coating and serum. Flow cytometry analysis shows that the phenotype of these cells on the uncoated Surface A is equivalent to that on the coated surfaces.

Cells cultured on CELLstart-coated vessels and uncoated Surface A vessels were differentiated into chondrocytes, adipocytes, and osteocytes. Manufacturers protocols were followed for differentiation and they are briefly described below.

Adipocyte Differentiation. Expanded MSCs were differentiated using the using the Gibco™ StemPro™ Adipogenesis Differentiation kit. Once cells reached 60-80% confluency, they were subcultured as described above. After cell dissociation and centrifugation, cells were re-suspended and seeded in an even distribution at 1×10⁴ cells/cm² in a 12 well Nunclon Delta culture vessel in StemPro™ MSC SFM XenoFree complete medium. After they reached 70-80% confluency, adipogenic differentiation was initiated using the Gibco StemPro Adipogenesis Differentiation kit. The cells were incubated at 37° C. with 5% CO₂ for at least 7 days with a complete media change every 3-4 days. Following this, the cultures were processed and stained using Oil Red O for visualization of oil droplets.

Chondrocyte Differentiation. Expanded MSCs were differentiated using the Gibco™ StemPro™ Chondrogenesis Differentiation Kit. Once cells reached 60-80% confluency, they were subcultured as described above. Once cells had been dissociated, they were centrifuged, counted, and re-suspended to a concentration of 1.6×10⁷ viable cells/mL. To generate micromass cultures, cells were seeded in 5-μL droplets of cell solution in the center of multi-well Nunclon Delta cell culture vessel. After cultivating micromass cultures for 2 hours under high humidity, warmed chondrogenesis media was added to culture vessels which were then incubated in 37° C. incubator with 5% CO₂. Cultures were fed every 2-3 days. After at least 14 days, cell masses were processed stained for mucin production using 1% Alcian Blue stain.

Osteocyte Differentiation. Expanded MSCs were differentiated using the Gibco™ StemPro™ Osteogenesis Differentiation Kit. Once cells reached 60-80% confluency, they were subcultured as described above. After centrifugation the cells were seeded at 5×103 cells/cm2. Cells were incubated in a 37° C. incubator with 5% CO2 in StemPro™ MSC SFM XenoFree complete medium till they reached 50% confluency. Following this, the media was replaced with complete, pre-warmed Osteogenesis Differentiation Medium. Media change was done every 2-3 days. After a 21-day differentiation period, the cells were fixed in formaldehyde and stained with Alizarin Red S solution for visualization of calcium deposits.

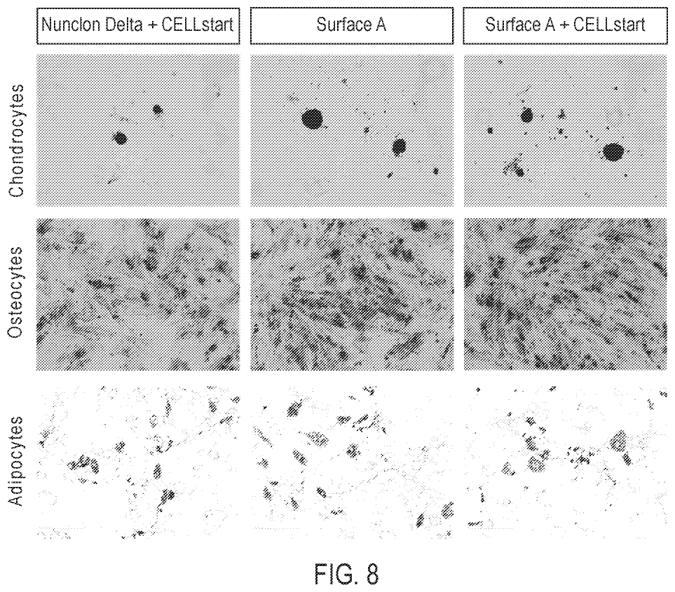

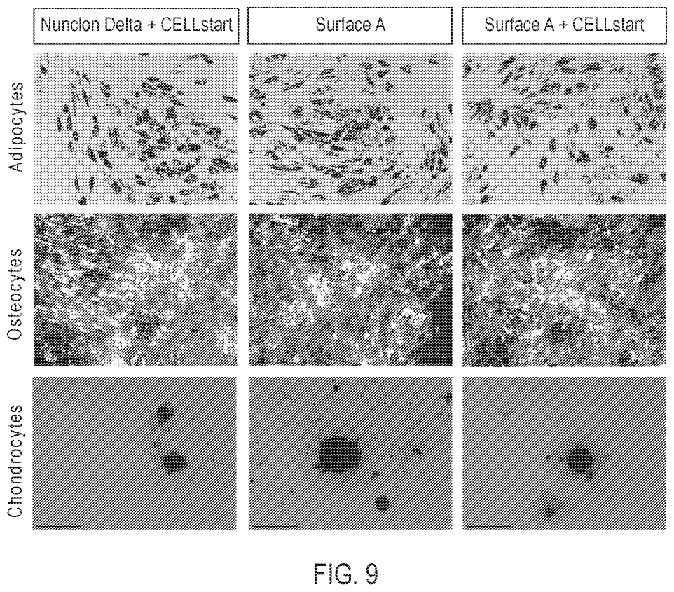

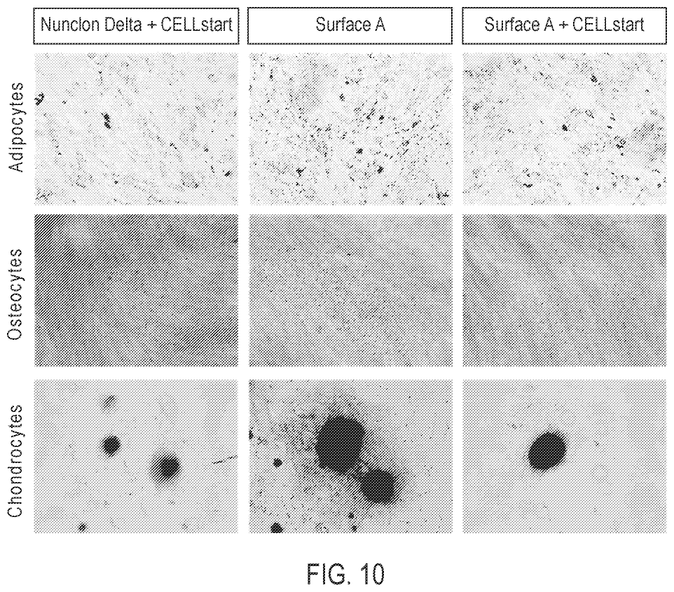

The expanded MSC populations were capable of differentiating to chondrocytes, osteocytes, and adipocytes. Whether cells were grown on a CellSTART-coated surface or an uncoated Surface A, there was very little qualitative difference between cell appearance and staining (FIGS. 8-10). Cells that were cultured on the Delta surface coated with CELLstart showed the expected cell differentiation results and serve as a positive control. Cells grown on Surface A without a coating look like the cells grown on the coated Delta surface. As shown in FIG. 8, bone marrow derived cells cultured on the uncoated Surface A in the absence of serum are capable of differentiating into chondrocytes, osteocytes, and adipocytes. As shown in FIG. 9, staining of the cells revealed that the adipose derived MSCs differentiated normally on uncoated Surface A despite the absence of a coating and absence of serum in the medium. As shown in FIG. 10, the Wharton's jelly derived MSCs grown in the absence of serum on the uncoated Surface A differentiated into adipocytes, osteocytes, and chondrocytes as shown by staining with Oil Red O, Alizarin Red S, and Alcian Blue respectively.

Human mesenchymal stem cells are normally cultured in the presence of serum and/or on an extracellular matrix coating on the cell culture vessel. Such culture conditions are not ideal as they can complicate, limit or prevent subsequent uses of MSCs grown under these conditions. Surface A is capable of supporting hMSC attachment, cell proliferation, and cell differentiation in the absence of serum and a coating. Culturing on the Surface A in the absence of serum did not significantly affect cell doubling time or cell viability in hMSCs derived from adipose, bone marrow, and Wharton's Jelly. Cells grown on the surface provided herein display cell surface markers CD90, CD73, CD105, and CD44 indicating they are still pluripotent. These cells are also capable of differentiating into adipocytes, osteocytes, and chondrocytes. Serum-free growth on uncoated Surface A has produced similar results in cell expansion with maintenance of pluripotency and differentiation capability have been obtained using human MSCs harvested from over 40 different donor individuals. The surface provided herein and its molecular content provide a cell culture surface that is conducive to hMSC growth and differentiation without serum and a coating. Accordingly, the surface provided herein has the potential to provide cell therapy grade hMSCs to treat disease in patients.

What is claimed is:

1. A method of expanding a mesenchymal stem cell population, the method comprising:
i. obtaining a plurality of mesenchymal stem cells (MSCs); and
ii. culturing the MSCs in serum-free growth medium on a surface, wherein the surface lacks a cell adhesive coating and lacks a feeder cell layer;
thereby generating an expanded MSC population.

2. The method of claim 1, wherein the surface contains at least about 0.9% N, has a sum of O and N of greater than or equal to 19.0% and has a static sessile contact angle of at least about 14 degrees.

3. The method of claim 2, wherein the surface contains about 0.9% to about 3.2% N, has a sum of O and N of about 19% to about 35% and has a sessile contact angle of about 14 to about 65 degrees.

4. The method of claim 2, wherein the surface contains about 1.3% to about 2.8% N, has a sum of O and N of about 22% to about 29% and has a sessile contact angle of about 17 to about 55 degrees.

5. The method of any one of claims 1-4, wherein the growth medium is serum-free.

6. The method of any one of claims 1-5, wherein the growth medium is animal origin-free medium and/or xeno-free medium.

7. The method of any one of claim 1-6, wherein the growth medium is selected from StemPro™ MSC serum-free medium, StemPro™ MSC XenoFree serum-free medium, MesenPRO RS™ medium, Dulbecco's Modified Eagle Media (DMEM), DMEM/F12, DMEM low glucose, DMEM high glucose, Advanced DMEM/F12; MSC NutriStem® XF medium, RPMI 1640 medium, Minimum Essential Medium α (MEMα), Iscove's Modified Dulbecco's medium (IMDM), and Basal Medium Eagle (BME).

8. The method of any one of claims 1-7, wherein the MSCs are isolated from bone marrow, adipose tissue, dermis, placenta, umbilical cord, amniotic fluid, synovial fluid, synovial membrane, deciduous teeth, or skeletal muscle.

9. The method of any one of claims 1-8, wherein the MSCs are human MSCs, non-human primate MSCs, murine MSCs, rat MSCs, pig MSCs, goat MSC, or sheep MSCs.

10. The method of any one of claims 1-9, wherein prior to the culturing of (ii) the plurality of MSCs are cultivated in a culture vessel comprising an adhesive coating on the culture vessel surface.

11. The method of claim 10, wherein the adhesive coating comprises extracellular matrix.

12. The method of any one of claims 1-11, wherein the culturing comprises growing the MSCs in growth medium on the surface through at least 3 passages.

13. The method of any one of claims 1-12, wherein the culturing comprises growing the MSCs in growth medium on the surface through at least 6 passages.

14. The method of any one of claims 1-13, wherein the surface is modified using a plasma treatment.

15. The method of any one of claims 1-14, wherein the surface comprises polystyrene, cyclic olefin polymer, cyclic olefin copolymer, polyolefin, polycarbonate, polymethyl methacrylate, styrene acrylonitrile copolymer, and mixtures and copolymers therefrom.

16. The method of any one of claims 1-14, wherein the surface comprises plasma-treated polystyrene.

17. The method of any one of claims 1-16, wherein the surface is part of a vessel or matrix.

18. A method of differentiating mesenchymal stem cells expanded according to the method of any one of claims 1-17, comprising incubating the expanded MSC population under conditions allowing the differentiation of said cells.

19. The method of claim 18, wherein the expanded MSC population is cultured under conditions allowing the differentiation of said cells into cells selected from the group consisting of osteoblasts, adipocytes, and chondrocytes.

20. A method for editing a genome in an MSC, the method comprising:
i. obtaining an MSC that was expanded using the method of any one of claims 1-17; and
ii. editing the genome of the MSC.

21. The method of claim 20, wherein the genome is edited using one of more genome editing reagents selected from a zinc-finger nuclease (ZFN), transcription activator-like effector nuclease (TALEN), meganuclease, and a clustered regularly interspaced short palindromic repeat (CRISPR) associated protein.

22. A method of treating a subject in need of a therapy, the method comprising:
i. obtaining a plurality of mesenchymal stem cells (MSCs); and
ii. expanding the MSCs in serum-free growth medium on a surface, wherein the surface lacks a cell adhesive coating and lacks a feeder cell layer; and
iii. transferring the expanded MSCs to the subject, thereby treating the subject.

23. The method of claim 22, wherein the surface contains at least about 0.9% N, has a sum of O and N of greater than or equal to 19% and has a static sessile contact angle of at least about 14 degrees.

24. The method of claim 23, wherein the surface contains about 0.9% to about 3.2% N, has a sum of O and N of about 19% to about 35% and has a sessile contact angle of about 14 to about 65 degrees.

25. The method of claim 24, wherein the surface contains about 1.3% to about 2.8% N, has a sum of O and N of about 22% to about 29% and has a sessile contact angle of about 17 to about 55 degrees.

26. The method of any one of claims 22-25, wherein the plurality of MSCs are derived from the subject.

27. The method of any one of claims 22-26, further comprising genetically modifying the MSCs prior to transferring the MSCs to the subject.

28. The method of any one of claims 22-27, wherein the subject is human.

29. A method for improving engraftment potential of a population of mesenchymal stem cells (MSCs), the method comprising:
i. obtaining a plurality of mesenchymal stem cells (MSCs); and
ii. expanding the MSCs in serum-free growth medium on a surface containing:
at least about 0.9% N, a sum of O and N of greater than or equal to 19% and having a static sessile contact angle of at least about 14 degrees, wherein the surface lacks a cell adhesive coating and lacks a feeder cell layer;
thereby improving engraftment potential of the expanded population of MSCs.

30. The method of claim 29, wherein the surface contains about 0.9% to about 3.2% N, has a sum of O and N of about 19% to about 35% and has a sessile contact angle of about 14 to about 65 degrees.

31. The method of claim 30, wherein the surface contains about 1.3% to about 2.8% N, has a sum of O and N of about 22% to about 29% and has a sessile contact angle of about 17 to about 55 degrees.

32. Use of an expanded population of MSCs generated according to a method of any one of claims 1-17 for the preparation of a medicament.

33. A population of mesenchymal stem cells (MSCs) expanded using the method of any one of claims 1-17 for use in a medicament to treat a subject.

34. The population of MSCs of claim 33, wherein the MSCs were derived from the subject to be treated prior to expansion.

35. The population of MSCs of claim 33 or claim 34, wherein the MSCs are genetically modified.
